(19) United States
(12) Patent Application Publication
(10) Pub. No.: US 2023/0381092 A1
(43) Pub. Date: Nov. 30, 2023

(54) Method for obtaining a purified substance from bee venom and anti-ageing cosmetic product comprising the purified substance

(71) Applicant: Hugo Patricio MORAGA FUENTES, Puerto Varas (CL)

(72) Inventor: Hugo Patricio MORAGA FUENTES, Puerto Varas (CL)

(21) Appl. No.: 18/249,689

(22) PCT Filed: Oct. 18, 2021
(86) PCT No.: PCT/CL2021/050102

§ 371 Date: Apr. 19, 2023

(30) Foreign Application Priority Data
Oct. 19, 2020 (CL) 2699-2020

(51) Int. Cl. A61K 8/98; A61Q 19/08

(52) U.S. Cl. CPC A61K 8/987; A61Q 19/08; A61K 2800/80

(57) ABSTRACT

The present invention relates to a method for separating and purifying melittin (Mn), apamin (Ap), mast cell degranulation (MCD) peptide and other components present in bee venom (BV). The method allows the beneficial components of the venom to be retained in the purified BV substance and allergens to be eliminated, using ultrafiltration and silica gel chromatography. The combination of the two techniques eliminates allergenic and harmful components, such as the enzymes phospholipase A2 (PLA2) and hyaluronidase (HYA), and a purified substance (referred to as “PDA”) is obtained from the venom. The invention also relates to an anti-ageing cosmetic product comprising the purified substance, wherein the cosmetic product is selected from a cosmetic serum.

The present invention relates to a method for obtaining a purified product (arbitrarily called “PDA”) from bee venom (BV, for its acronym in English “Bee Venon”) that comprises peptides, carbohydrates and minerals, that is, the beneficial components of the poison, using Ultrafiltration and Silica Gel Chromatography. The present invention also refers to an anti-aging cosmetic product that comprises PDA as an active ingredient in the formulation of a Serum with anti-free radical and skin protective properties.

### BACKGROUND

Skin aging can be intrinsic (physiological factors) and/or extrinsic (environmental factors), and this process negatively impacts skin functions by altering its protective barrier function (Rodan et al. 2016. Skincare Bootcamp: The Evolving Role of Skincare. Plastic and Reconstructive Surgery—Global Open, 12S—p e1152). Intrinsic skin aging occurs naturally with advancing age, triggering the appearance of wrinkles and dryness (Raine-Fenning et al. 2003. Skin aging and menopause: implications for treatment. Am J Clin Dermatolm 4:371-378); on the other hand, extrinsic aging is due to environmental factors and may even be due to lifestyle (smoking) and/or lack of balanced nutrition (Farage et al. 2008. Intrinsic and extrinsic factors in skin aging: a review Int J Cosmet Sci, 30:87-95). In both types of aging, oxidative damage caused by the overproduction of free radicals plays an important role (US 2010/0035098 A1: Bonte et al. 2019. Skin Changes During Ageing. Subcell Biochem, 91:249-280). The signs of aging (wrinkles, fine lines of expression, dark spots, loss of elasticity and luminosity) have been combated for years through invasive or non-invasive techniques such as: surgery, laser treatments, injections with growth factors and the use of cosmetic products with active components focused on improving the appearance of the skin (Rodan et al. 2016. Skincare Bootcamp: The Evolving Role of Skincare, Plastic and Reconstructive Surgery—Global Open, 12S—p e1152; Mukherjee et al. 2001. Bioactive compounds from natural resources against skin aging. Phytomedicine, 19: 64-73: Emerald & Kumar 2016. Perspective of Natural Products in Skincare. Pharmacy & Pharmacology International Journal, 4:1-3). Cosmetic treatments include the use of creams and lotions to reduce or prevent the effects of skin aging and are also a complement to more invasive treatments (Fabi et al 2017. Optimizing Facial Rejuvenation with a Combination of a Novel Topical Serum and Injectable Procedure to Increase Patient Outcomes and Satisfaction. J Clin Aesthet Dermatol, 10:14-18). Such treatments are designed to improve the appearance and condition of dry or scaly skin and/or to heal skin that has been irritated by exposure to chemicals, wind or sunlight, among other potential irritants. BV is found used in cosmetic products (KR20120038699A, KR101394817B1. JP2012533617A) for anti-wrinkle (KR101394817B1) and anti-cane (KR20120038699A, JP2012533617A) purposes. In anti-acne studies, BV is used due to its anti-inflammatory and antibacterial action, it should be noted that BV in the aforementioned patent documents does not have any prior purification procedure that removes PLA2 and HYA enzymes, and is considered its use within the formulation in the range of 0.001%-0.05% w/w (KR20120038699A), and also 0.0001%-10% w/w. In addition. KR101394817B1 considers the use of a second raw material derived from bees, which within the formulation considers royal jelly at 0.1% w/w. But the joint use of BV with other bee-derived raw materials that may also contribute to the desired cosmetic effect has not been disclosed or suggested in the prior art. In KR101394817B1, for anti-wrinkle cosmetic purposes, they use unpurified BV or a BV extract obtained by solvent extraction in the range of 0.001-5% w/w, they determine that BV can have a pro-collagen effect like vitamin C.

The BV produced by worker bees contains many active components including—proteins, peptides, enzymes, sugars, amino acids, volatile compounds and minerals- (Moreno & Giralt 2015. Three valuable peptides from bee and wasp venoms for therapeutic and biotechnological use: melittin, apamin and mastoparan. Toxins (Basel), 7:1126-1150). Apamin (Ap: 2.027 KDa), melittin (Mn: 2.85 KDa), Phospholipase A2 (PLA2: 18.964 KDa), mast cell degranulating peptide (MCD: 2.6 KDa), and Hyaluronidase (53.87 KDa). Where the most abundant is Melittin (Mn), representing between 40 to 50% in dry weight of apitoxin. Within these compounds there are two powerful allergens and destroyers of skin cells, such as PLA2 and HYA, both enzymes represent between 10-12% and between 1-2% in dry weight of apitoxin, respectively (Bogdanov et al. 2017. Bee Venom: Composition. Health, Medicine: A Review. Bee Product Science, 1-16). Several studies have shown the therapeutic potential of these components in the treatment of human inflammatory diseases. Central nervous system diseases, such as Parkinson's disease, Alzheimer's disease, and amyotrophic lateral sclerosis and other rheumatic conditions (Wehbe et al. 2019. Bee Venom: Overview of Main Compounds and Bioactivities for Therapeutic Interests. Molecules, 24:2997). In addition, BV, like other animal venoms, has also been shown to be beneficial against ovarian and prostate cancer, as well as HIV (Wehbe et al. 2019. Bee Venom: Overview of Main Compounds and Bioactivities for Therapeutic Interests Molecules 24:2997; Duffy et al 2020. Honeybee venom and melittin suppress growth factor receptor activation in HER2-enriched and triple-negative breast cancer, npj Precis One. 4: 24). Studies indicate that peptides present in BV such as Mn and Ap have skin-restoring and photoprotective properties, generate cell migration and promote cell renewal, among others (Han et al, 2013. Effects of honeybee (Apis mellifera) venom on keratinocyte migration in vitro. Pharmacogn Mag. 9:220-226: Wehbe et al., 2019. Bee Venom: Overview of Main Compounds and Bioactivities for Therapeutic Interests. Molecules, 24:2997: Lee et al., 2014. The protective effects of melittin on Propionibacterium acnes-induced inflammatory responses in vitro and in vivo. J Invest Dermatol, 134: 1922-1930; Ovcharov et al., 1976. Antinflammatory effects of pamin. Toxicon, 14:441-447). However, its use is limited precisely due to the adverse effects due to the presence of PLA2 and HYA (Pascoal et al. 2019. An overview of the bioactive compounds, therapeutic properties and toxic effects of apitoxin. Food Chem Toxicol, 134:110864; Wehbe et al., 2019. Bee Venom: Overview of Main Compounds and Bioactivities for Therapeutic Interests. Molecules, 24:2997; Kurek-Górecka et al. 2020. Bee Products in Dermatology and Skin Care. Molecules, 25, 556.). The FDA has approved the use of purified BV fractions from Apis mellifera, Apitox® (Apimeds, Inc., Seongnam-si, Korea: WO2011041865), to relieve pain and swelling associated with rheumatoid arthritis, tendonitis, bursitis, and multiple sclerosis, being possible its use in gels or injection (Seo et al, 2014. BK, Bee venom acupuncture, NSAIDs or combined treatment for chronic neck pain: Study protocol for a randomized, assessor-blind trial. Trials.; 15:132). Another relevant aspect is the ability of some peptides to penetrate the stratum corneum, where the ability of Mn to cross skin barriers has been studied (Chenet al., 2016. Melittin, the Major Pain-Producing Substance of Bee Venom Neurosci Bull, 32:265-272).

Due to the above, interest has arisen in separating and purifying the active components from the BV, in order to use them in the formulation of a cosmetic product that can improve and correct skin imperfections and provide nutrition.

The purification techniques of the active components from BV are mainly focused on the purification of Mn (CN101089017A, CN109045281 A, KR100744755B1, KR20170066943A, KR20150049865A, RU2066997C1) and other components are focused on only removing those with higher molecular weight (such as PLA2 and HYA). CN1025261 16A, KR101364506B1, KR101667544B1. KR101734093B1, KR20140006235A, WO2014196674A1). The techniques that focus on purifying Mn eliminate components that are part of the BV and that as a whole also provide biological properties. In CN109045281 A, a solvent precipitation using n-butanol and ethanol is used to remove impurities and extract BV in the aqueous phase. The thick aqueous phase of BV is subjected to size exclusion chromatography, where it is separated under acidic conditions at pH 3-4 (Sephadex G 20). This methodology is much more analytically complex than the present invention, where under pH 4 it is favored that the protein is in the form of a monomer and is separated by molecular exclusion chromatography. With this methodology, Mn was purified to be used in a pharmaceutical preparation for endometriosis. In CN101089017A they purify Mn through a succession of steps that consist of removing particulate material by filtering through filter paper, then precipitation in ethanol followed by extraction with n-butanol and ammonium hydroxide, concentration of the extract and a second precipitation with acetone, where the precipitate is dissolved in buffer urea acetate. After these steps of extraction and precipitation by solvent, cation exchange chromatography (CM-Sepharose) and size exclusion chromatography (Sephadex G25) were performed, where after each chromatography desalting of the collected fractions (Sephadex G10) had to be performed, because the mobile phase used in both chromatographic techniques consisted of saline buffers. Finally, in CN101089017A they manage to obtain from 20 g of BV. a final lyophilisate of 9.4 g of Mn (% yield of 47%) with a degree of purity level electrophoresis. Reverse phase chromatography has also been used to purify Mn, in KR20170066943A they use C-4 resin and separate the BV components using a gradient with a water-ethanol mixture, where they determine a recovery rate of Mn in a solution of purified from BV at 50 mg/mL obtaining 88.5%, finally; the purity of 98.2%.

Other alternative ways focused on eliminating PLA2 and HYA, use heat inactivation of proteins in order to eliminate the activity of PLA2 (10 to 12% w/w) and HYA (1.5-2% w/w) (Lokeshwari, & Shantibal 2010. A Review on the Fascinating World of Insect Resources: Reason for Thoughts. Psyche: A Journal of Entomology, 10: 1155.); however, this strategy does not prevent the possible development of an allergenic response by the host and the denaturation of the peptides present (KR101667544B1). Chromatographic techniques have also been used to remove PLA2 and HYA, in CN1025261 16A they use solvent precipitation in conjunction with exclusion chromatography (Sephadex G50) in order to remove macromolecules such as PLA2 and HYA. To begin the purification process, the BV is dissolved in 5-10 times more water depending on the proportion of BV to be dissolved, followed by centrifugation to remove particulate material. The aqueous fraction recovered from the centrifugation is subjected to solvent precipitation and the precipitate obtained is dissolved in the buffer to be used for size exclusion chromatographic separation. In CN102526116A they manage to reduce the content of macromolecules in the BV from 16% (dry weight of BV) to a content <1.5% and with a content of Mn >76%. Therefore, with this strategy there is still a content of PLA2 and HYA, despite obtaining a good yield for Mn. In another invention (KR100744755B1) they manage to purify Mn also using solvent precipitation and size exclusion chromatography with Sephadex G50 resin, obtaining three characteristic factions, but, to achieve the purification of Mn, an extra step is performed by subjecting the fraction containing Mn (of the previous three) to size exclusion chromatography with Sephadex G25, finally obtaining 1.08 g of Mn from 5 g of BV (yield of 21.6%), this strategy to purify Mn has a low yield compared to the other methods described.

The tetramelization of Mn, a peptide of 26 amino acids, occurs when: the pH is raised from 4.0 to 9.5; increases ionic strength; the temperature is around 37° C. The stability of this peptide is maintained between 35.5 and 43° C. in a pH-dependent manner, unfolding above and below that temperature. Therefore, methodologies that avoid a destabilization of the components are very important, if it is to retain all the biological properties. Other proposed methods involve the use of ultrafiltration, as indicated in WO2014196674A1, where the BV solution dissolved in water is filtered on an ultrafiltration membrane (10 kDa cut-off size) to remove PLA2 and HYA. However, although Mn is 2.85 KDa in size, at pH 7.2 it is in the tetramer form (12.5 KDa), being retained on the 10 KDa filter and only a very small amount is filtered. With the method proposed in the present invention, the concentrate obtained by ultrafiltration presents Mn, PLA2 and HYA, which due to their molecular weight are retained, the above contrasts with the results obtained from the methodology described in WO2014196674A1, it makes present the need that this fraction is separated through another analytical technique.

The present invention consists of a chromatography with Silica gel 60 that allows the proteins present to be separated based on their differences in polarity. This technique has little exploration in the field of purification (Gabriel et al. 1976. Preparative high-performance liquid chromatography applied to peptide synthesis. Journal of Chromatography A, 129: 287-293) or pre-purification of peptides (Harada et al. 1988. Improved method for purification of toxic peptides produced by cyanobacteria. Toxicon. 26: 433-439.) natural or synthetic, and resulted in good performance and easy to use. Furthermore, according to the present invention, the method for preparing a purified BV has a first step of preparing a BV powder that is diluted in water, a second step of filtration to remove impurities (0.45 and 0.22 pm), a third step of ultrafiltration and a fourth step to remove harmful components and recovery of Mn, Ap and traces of MCD by Silica Gel chromatography. According to the method for preparing a purified BV in the present invention, PLA2 and HYA enzymes induce a strong allergic reaction and are removed by a combination of “Silica gel G60 ultrafiltration”. The evaluation of the elimination of allergens (PLA2 and HYA) is monitored by enzymatic assays, where the activity of both enzymes in the samples is determined. To evaluate PLA2. EnzChek® Phospholipase A2 Assay (Invitrogen) and a turbidometric assay described by Enzymatic Assay of Hyaluronidase (Sigma-Aldrich, St. Louis, MO, USA) were used to evaluate hyaluronidase activity throughout the process and also to determine the absence of them by HPLC.

### BRIEF DESCRIPTION OF THE INVENTION

The present invention relates to an analytical method that allows the components present in BV to be separated (FIG. 1). For this, the complete BV is used, which is solubilized in deionized water and filtered using a 0.45 and 0.22 pm membrane to remove particulate material. This BV is then characterized to evaluate its protein composition through reverse phase high performance chromatography (HPLC) (LC 250×4.6 mm, LUNA C18 5 pm, 100 Å, Phenomenex). For separation, an ultrafiltration system is used, Amicon® Ultra centrifugal (10 KDa pore), where low molecular weight components are separated by centrifugation and molecules over 10 KDa are retained. After the separation process, a BV filtrate (FBV) and a BV concentrate (retained in the filter) are obtained. To evaluate protein content, purity of FVB and CBV are analyzed by HPLC, SDS-PAGE, respectively. The presence of PLA2 and HYA is monitored through the measurement of enzymatic activity, using the EnzChek® Phospholipase A2 Assay (Invitrogen) and a turbidometric Enzymatic Assay of Hyaluronidase assay (Sigma-Aldrich, St. Louis, MO, USA). In the FBV low molecular weight components such as Ap, MCD, sugars, amino acids and Mn (<5%) are obtained without the presence of PLA2 and HYA and it is stored at 4° C. However, in the CVB this method drags Mn (>90%), Ap (<10%) and traces of MCD (traces: below detection limit), leaving all of PLA2 and HYA. Therefore, the CBV is subjected to Silica Gel (G60) chromatography, where it is possible to obtain the complete separation of Mn. MCD and Ap from the PLA2 and HAY components, which were analyzed by HPLC, SDS-PAGE and evaluation of enzymatic activity. The content of the peptides present and the performance of the methodology were quantified. The fraction obtained from the Silica gel, free of PLA2 and HYA, was combined with the FBV and lyophilized to later determine the final concentration and said fraction obtained was called PDA “purified from BV”. This PDA, obtained is the active ingredient for the preparation and formulation of a Serum for cosmetic purposes.

Likewise, the present invention is related to a cosmetic product that includes the purified product described in the previous paragraph, and its preparation method.

### BRIEF DESCRIPTION OF THE FIGURES

FIG. 1. Sample of bee venom (BV) of Apis Mellifera obtained from the hives of using traps with electro-stimulation (Ketitlen, Colombia).

FIG. 2. HPLC chromatogram of BV. BV sample of Apis Mellifera bees Carnica type from Puerto Varas diluted in water. The major peaks represent: (1) Ap (Apamina); (2) MCD (Mast Cell Degranulating Peptide); (3) PLA2 (Phospholipase A2) and (4) Mn (Melittin).

FIG. 3. Tricine gel -SDS-PAGE 16% of samples separated using the Amicon® Ultra centrifugal Filter system of 10 Kda for 40 min. 1) Molecular weight marker; 2) sample Concentrated fraction; 3) Filtered fraction.

FIGS. 4A and 4B. HPLC chromatograms BV fractions. FIG. 4A: HPLC of the CBV sample obtained by Amicon®; shows the peaks for Mn (4), PLAA2 (3), MCD (2) and Ap (1). FIG. 4B: Chromatogram of FBV obtained by Amicon®, the peaks represent Mn (4), MCD (2) and Ap (1).

FIG. 5. Silica gel G60 chromatography. Elution profile of the fractions monitored by absorption at 280 nm.

FIG. 6. HPLC chromatogram of purified BV (PDA). The chromatographic profile of the PDA is shown, where Mn (4) is observed; MCD (2) and Ap (1).

FIGS. 7A-7D. Effect of PDA and BV on Staphylococcus aureus and Staphylococcus epidermis microorganisms. Viability of S. aureus and S. epidermis. FIG. 7A: % Viability versus time in hours when S. aureus is exposed to 8 pg/mL PDA and BV. FIG. 7B: A decrease in viability is observed when S. aureus is exposed to Kanamycin (positive control), BV and PDA. FIG. 7C: % Viability versus time in hours when S. epidermis is exposed to 8 pg/mL PDA and BV. FIG. 7D: A decrease in viability is observed when S. epidermis is exposed to Kanamycin (positive control), BV and PDA.

FIGS. 8A and 88. Effect of PDA and BV on the viability of HDFa cells. The cells were incubated in a range of 1 pg/mL to 100 pg/mL in incubated for 3 hours (FIG. 8A) or 24 hours (FIG. 8B).

FIG. 9. Wound closure test. The effect of PDA and BV Ha on HDFa cells over time is evaluated. ***=P-value<0.0001.

FIG. 10. Effect of PDA on wound closure in HDFa cells. Control cells and PDA cells were incubated and photographed at different times. The red line shows the wound caused by the tip.

FIGS. 11A and 11B. Percentage of release of the enzyme Lactate Dehydrogenase (LDH). FIG. 11A: HDFa cells are exposed to an MOI of 5 of S. aureus and different concentrations of MS extracts for 24 hours. FIG. 11B: HDFa cells when exposed to an MOI of 5 of S. epidermis and different concentrations of MS extracts for 24 hours.

FIG. 12. Percentage COX-2 inhibition by PDA and BV. Values expressed as mean±SD where, n−3, statistical significance performed by ANOVA followed by Tukey's multiple comparison test. (#P<0.0001 comparing between both concentrations, P<0.0001 comparing with the control at the same concentration).

FIGS. 13A-13C. Percentage of formation of reactive oxygen species (ROS). FIG. 13A. they are exposed for 3 hours to S. aureus and different concentrations of PDA. FIG. 13B. When treated for 3 hours with different concentrations of PDA, and later for 3 hours with S. aureus. FIG. 13C. When cells are treated with different concentrations of PDA and BV. “ ”=P-value<0.0001.

FIGS. 14A-14C. Percentage of formation of reactive oxygen species (ROS). FIG. 14A. They are exposed for 3 hours to S. epidermis and different concentrations of PDA and BV. FIG. 14B. When treated for 3 hours with different concentrations of PDA and BV, and later for 3 hours with S. epidermis. FIG. 14C. When cells are treated with different concentrations of PDA and BV. ****=P-value<0.0001.

### DETAILED DESCRIPTION OF THE INVENTION

The present invention relates to a method for purifying bee venom comprising the following steps:
a) separate the beneficial components present in complete bee venom (BV) from the allergenic components, solubilizing it in deionized water and then filtering it through a 0.45 and 0.22 pm membrane to remove particulate matter;
b) characterize the protein composition of the filtrate resulting from step a) by high performance chromatography (HPLC), in reverse phase:
c) separating the low molecular weight components of the filtrate characterized from step b) by ultrafiltration, including said low molecular weight components Ap. MCD, sugars, amino acids and Mn and evaluating the absence of PLA2 and HYA by SDS-PAGE electrophoresis, HPLC and enzyme assays. Enzymatic assays evaluate enzymatic activity (EnzChek® Phospholipase A2 Assay (Invitrogen) and Enzymatic Assay of Hyaluronidase (Sigma-Aldrich, St. Louis, MO, USA), and subsequent storage at 4° C.;
d) determine the amount of Mn in the concentrate that is retained in the filter (CBV) of step c) by HPLC, SDS-PAGE, which is subsequently purified by Silica Gel G60 column chromatography, and the purity of Mn is determined, that is, free of PLA2 and HAY by HPLC, SDS-PAGE and measurement of enzymatic activity using EnzChek® Phospholipase A2 Assay (Invitrogen) and a turbidometric Enzymatic Assay of Hyaluronidase assay (Sigma-Aldrich, St. Louis, MO, USA), in the fractions resulting from silica gel chromatography, quantifying the content of peptides present and yield,
e) joining the fraction obtained from silica gel free of PLA2 and HYA with the FBV, lyophilized and the final concentration is determined.

The purified obtained was called PDA “purified from BV”. This PDA is the active ingredient for the preparation and formulation of a Serum for cosmetic purposes.

Likewise, the present invention is related to a cosmetic product that includes the purified product described in the previous paragraph, and its preparation method.

In addition, according to the present invention, the method for preparing a purified BV, optionally comprises in step a), the preparation of bee venom powder that is subsequently diluted in water, to then carry out a filtration to remove impurities (0.45 and 0.22.m), and subsequently ultrafiltration and removal of harmful components PLA2 and HYA and recovery of Mn and peptide components (Ap and MCD) by Silica Gel chromatography. According to the method for preparing a purified BV in the present invention, PLA2 and HYA, which induce a strong allergic reaction, are removed by a combination of ultrafiltration with silica gel G60, the evaluation of allergen removal is monitored, by specific enzyme assays described throughout the process and HPLC.

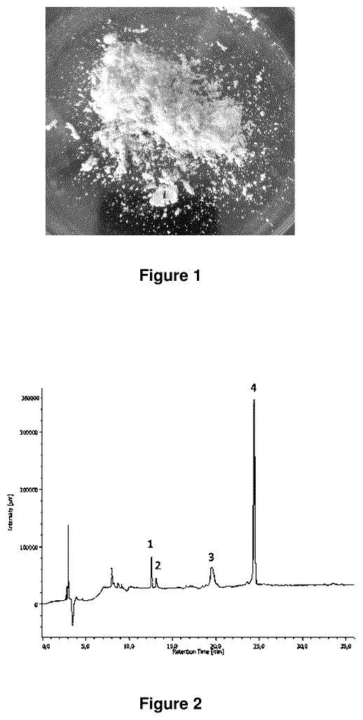

Example 1: Characterization and purification of the BV components: In FIG. 1, the BV (2 g) collected from the hives is shown. To obtain the BV, electro-stimulation (Ketitlen, Colombia), then it was dissolved in sterile deionized water at a concentration of 4 mg/mL, it was filtered using a 0.45 and 0.22.m membrane to remove debris and impurities present. Using reverse phase HPLC chromatography (250×4.6 mm, LUNA C185 pm, 100 Å, Phenomenex), the crude extract of BV from Apis Mellifera was characterized in terms of its main protein components (FIG. 2). The total concentration of the protein content of BV in each stage of the process was determined by HPLC and by the bicinchoninic acid (BCA) method, which consists of a compound capable of forming an intense purple complex with Cu¹⁺ ions in an alkaline medium. (Smith, P K, et al. 1985. Measurement of protein using bicinchoninic acid. Anal Biochem 150:76-85) and read at 562 nm, using a bovine serum albumin (BSA) standard curve.

TABLE 1
Concentration proteins presents in whole
BV in mg/ml determined by HPLC

| Compound | μg/ml | % p/p |
|---|---|---|
| Mn | 38.1 ± 25.5 | 38.2 ± 2.6 |
| Ap | 19.95 ± 2 | 2 ± 0.2 |
| MCD | 9.95 ± 1 | 1 ± 0.1 |
| PLA2 | 199.9 ± 6.5 | 12 ± 1.3 |
| HYA | ND | ND |

ND, Not detected

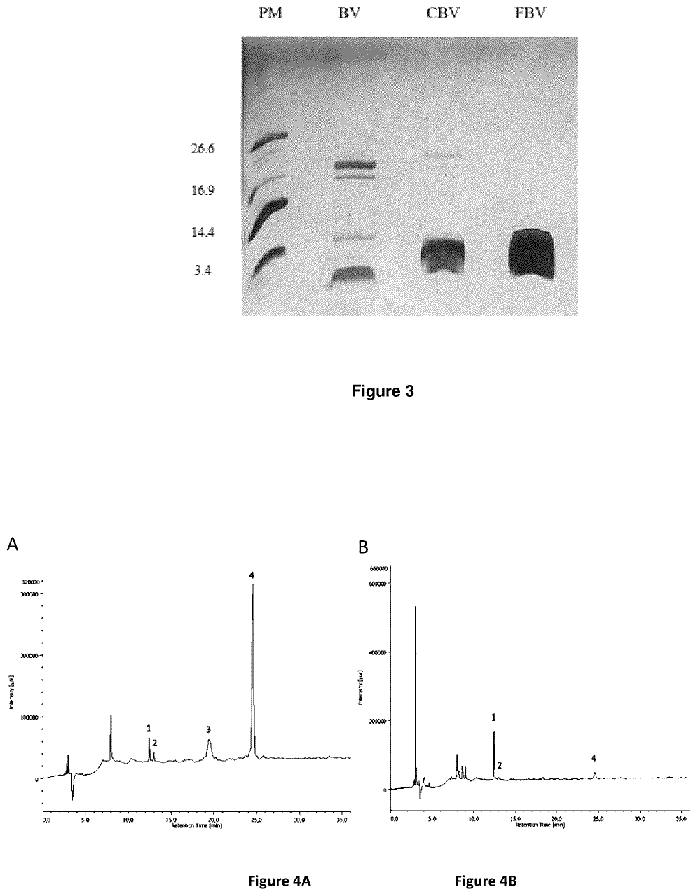

Example 2: Purification of the bloactive components of apitoxin: For the purification, 4.0 mL of the filtered extract were used at a concentration of 4 mg/mL, and were passed using the 10 KDa Amicon® Ultra centrifugal Filter ultrafiltration device. Device that has a membrane that allows the separation of proteins or peptides with a molecular weight above 10 KDa, and allows proteins below that molecular weight to pass through. It was then centrifuged for 40 min at 40,000 g (4° C.). After centrifugation, a concentrated fraction (CBV: upper part) was obtained that contains proteins and molecules of size over 10 KDa and a filtered fraction called FBV (what passes through the filter), where low molecular weight proteins are found. The FBV and CBV obtained from Amicon® were lyophilized and analyzed by RP-HPLC and 16% SDS-PAGE gel electrophoresis to determine the presence of the protein in both fractions obtained. FIG. 3 shows that high PM proteins remained in the CBV and Mn (>90% of the total) and Ap (>2% of the total) and MCD (traces: below the detection limit) were dragged along, which was confirmed by HPLC (FIG. 4A) and in the FBV it can be verified that the Mn was not retained; however, PLA2 was completely removed (FIG. 4B). In addition, PLA2 and HYA activity was evaluated in the fractions (FBV and CBV) obtained using the EnzChek® Phospholipase A2 Assay kit from Invitrogen™ for the determination of PLA2. Briefly, a calibration curve was performed in a range of 0-10 units/mL of PLA2, which was incubated together with liposomes composed of DOPG and DOPC, which contain the fluorescent substrate BODIPYOPC-A2. The purified sample and VB are interpolated on the calibration curve and the percentage of enzyme remaining was calculated using Equation 1.

PLA2%=PLA2 in purified/PLA2 in raw poison×100 (equation 1)

To evaluate HAY activity, an Enzymatic Assay of Hyaluronidase turbidometric assay (Sigma-Aldrich, St. Louis, MO, USA) was performed. Briefly, 250 μl of the samples to be tested were mixed with 250 μl of enzyme diluent (20 mM sodium phosphate with 77 mM NaCl and 0.01% (w/v) BSA, pH 7.0 at 37° C.). The sample was allowed to stand for 10 min at 37° C. Then hyaluronic acid solution (500 μl) was added and mixed by shaking, followed by incubation at 37° C. for 45 minutes. After 45 min, 34 μl of mixture was added to 170 μl of acidic albumin solution and transferred to a 96-well plate. The solution was allowed to stand for 10 minutes at room temperature, and the measurement was performed at 600 nm. The percentage of HYA enzyme remaining was calculated using Equation 2.

HYA%=HYA in purified % HYA in raw poison×100 (equation 2)

In both cases, in the complete BV and in the CBV fraction, 100% activity was found for both enzymes, respectively, and 0% activity for both enzymes in FBV.

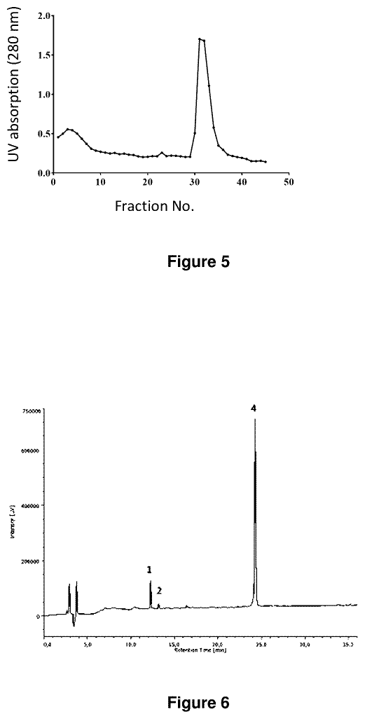

Example 3: Separation of melittin and peptide residues by silica gel. The CBV fraction contained Mn and traces of MCD and Ap, which were purified using a Silica gel G60 column. Silica gel is a polar adsorbent, slightly acidic, it has a powerful ability to absorb basic contents such as proteins that may be in the material to be purified. The 20 mL column was equilibrated with a mobile phase Butanol: Acetic acid: H₂O (4:3:1), the sample was loaded in the same mobile phase (2 mL) and then washed with 3 volumes of the same, previous mobile phase, then with 3 volumes of mobile phase in a ratio of 5:3:1, and finally 3 volumes of 100% methanol. (FIG. 5). As shown in FIG. 5, the fractions eluted from the silica Gel G60 column were monitored by spectrophotometric measurement in the UV region. From fraction 1 to fraction 17 approximately corresponds to the elution with the mobile phase 4:3:1, where the elution of PLA2 and HYA is observed, these are initially eluted given their large size and low acidity, therefore which does not interact with silica gel. From fraction 1 1 to approximately fraction 33 corresponds to the elution with the 5:3:1 mobile phase, where a decrease in the acidity of the medium occurs and the release of Mn and the other peptides begins (FIG. 5).

From fraction 34 to fraction 45, elution is performed with 100% MetOH, where a proton exchange with Silica occurs, generating a change in the interaction with Mn and Ap, which are released from the column. Subsequently, these fractions were concentrated in a rotary steamer at 40° C. The product obtained was resuspended in the FBV fraction (previously stored at 4° C.) thus obtaining the PDA, which was lyophilized at −80° C. (SpeedVac, Thermo Scientific®, model SC250EXP P2). In this way, a PDA is obtained that contains not only the peptides, but also the other constituents of BV are retained. To verify that the methodology used allowed us to separate the PLA2 and HYA enzymes from the other protein components, the already lyophilized PDA was dissolved in water to be evaluated for purity by HPLC (FIG. 6). In addition, to verify that the PDA was free of PLA2 and HYA, an enzymatic assay was performed to measure enzymatic activity using EnzChek® Phospholipase A2 Assay (Invitrogen™) and Enzymatic Assay of Hyaluronidase (Sigma-Aldrich, St. Louis, MO, USA), respectively. After the enzyme activity assay for both proteins, it was negative, as can be seen in FIG. 6 and Table 2, which summarizes all the fractions analyzed during the process. Table 3 shows the content of proteins present in the PDA.

TABLE 2
Phospholipase A2 (PLA2) and hyaluronidase
(HYA) enzymatic Activity

| Sample | PLA2% Activity | HYA % Activity |
|---|---|---|
| BV | 100 | 100 |
| CBC | 99.4 | ND |
| FVB | 0 | 0 |
| PDA | 0 | 0 |

TABLE 3
Protein concentration presents in
PDA in μg/g determined by HPLC

| Compound | μg/g | % p/p |
|---|---|---|
| Mn | 663.4 ± 13.4 | 66.34 ± 0.7 |
| Ap | 204.1 ± 3 | 20.41 ± 0.3 |
| MCD | 132.5 ± 8 | 13.25 ± 0.8 |
| PLA2 | 0 | 0 |
| HYA | ND | ND |

ND: Not Detected

### Example 4: Effect of PDA on Epithelial Microorganisms Microorganisms

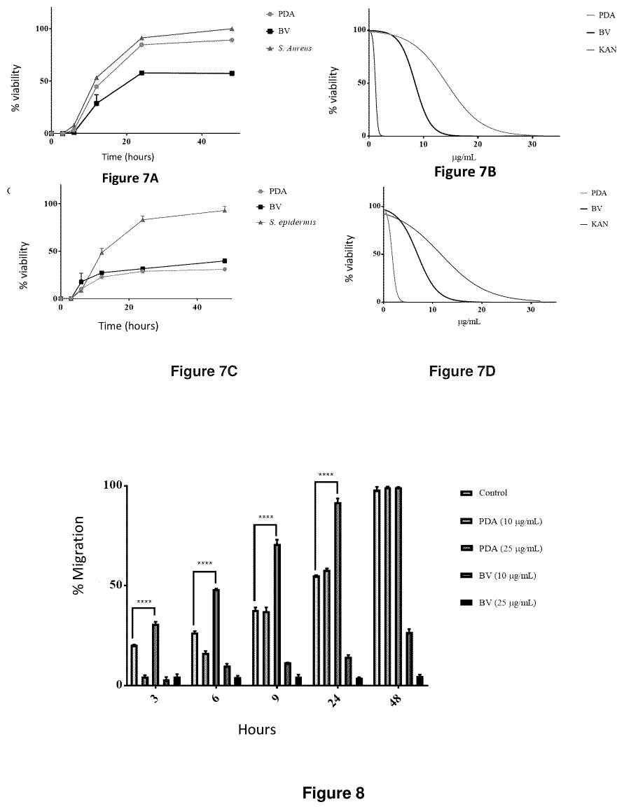

A) Cytotoxic effect of PDA on S. aureus and S. epidermis: In order to evaluate the inhibition of the growth of these bacteria by PDA. The concentrations considered for the formulation of the Serum are from 0.006 to 0.012%. Therefore, its use on the skin considering 0.2 mL is approximately 12 to 24 g of PDA, within this range a decrease in the viability of both microorganisms is observed as shown in FIG. 7. Although, BV has a greater effect on S. aureus than PDA. This increased effect may occur due to the presence of other components in BV: on the other hand, a greater effect of PDA on the inhibition of the growth of S. epidermis is observed. Therefore, PDA is effective in decreasing the growth of opportunistic epithelial pathogens.

### Example 5: Effects of PDA in a Skin Fibroblast Cell Model

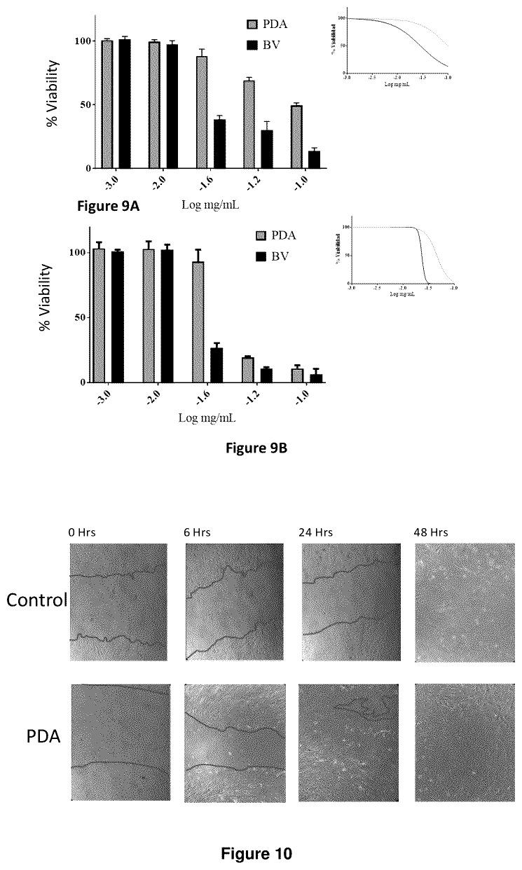

Example 5A: Cytotoxic Effect of PDA on HFDa Cells: It is very important to assess whether concentrations that affect the viability of bacteria have a damaging effect on human epithelial cells. For this, the optimal concentrations that do not cause damage to the cells were determined. Therefore, the tests were performed using a range of concentrations between 0 and 100/mL·BV was shown to be highly cytotoxic on cells in culture vs. PDA at concentrations of 25|g/mL, therefore, the complete BV is not suitable for direct use on the skin (FIG. 9).

Example 5B: Effect of PDA on the migration of HDFa cells. The ability of the PDA compound to induce cell migration was evaluated through a wound closure assay (Becker et al. 2014. Coagulase-negative staphylococci. Clinical Microbiology Reviews, 27:870-926). The cells were incubated with 10 and 25 ug/mL of PDA and BV. In addition, vehicle (ethanol) was used as a positive control. Cellular migration towards the wound was recorded by photographs under a microscope and taken at 0, 3, 6, 9, 24 and 48 hrs, from the start of treatment with the compounds (time 0 hrs). Images were analyzed with ImageJ 1.42q imaging software (National Institutes for Health. US). The percentage of cell migration was calculated with Equation 3:

% Migration=(Do−Dn)/Do×100 (equation 3)

where Dₒ is the average distance between cells produced by the wound and Dₙ is the average distance between cells at different times, from time 0. In Table 4 shows the migratory percentages of the HDFa cell line when exposed to different concentrations of PDA and BV between the initial time (0) and 48 hours.

TABLE 4
Effect of PDA and BV on HDFa cell migration.

| | | PDA (pg/mL) | | BV (pg/mL) | |
|---|---|---|---|---|---|
| Control | Time | 10 | 25 | 10 | 25 |
| 3 | 20.36 ± 0.14 | 4.54 ± 0.69 | 31.05 ± 0.85 | 3.01 ± 1.23 | 4.50 ± 1.16 |
| 6 | 26.60 ± 0.65 | 16.38 ± 0.92 | 48.19 ± 0.35 | 10.04 ± 0.86 | 4.10 ± 0.77 |
| 9 | 38.85 ± 1.22 | 37.34 ± 1.78 | 70.85 ± 2.14 | 11.53 ± 0.02 | 4.56 ± 0.95 |
| 24 | 55.03 ± 0.19 | 57.94 ± 0.70 | 91.73 ± 1.99 | 14.31 ± 0.93 | 4.05 ± 0.06 |
| 48 | 98.06 ± 2.84 | 99.30 ± 0.37 | 99.19 ± 0.23 | 26.86 ± 1.41 | 4.72 ± 0.72 |

*Values represent % migration over time in hours.

As seen in Table 4 and FIG. 10, the migration percentage is represented with respect to time and the effect of PDA and BV on this event. It is possible to observe how the PDA exerts a positive effect on the migratory capacity of the cells at concentrations of 25 pg/mL, at 9 hours of incubation there was a 70% closure of the wound compared to the control and at 24 hours a 92%. As an example of these tests, FIG. 10 shows images taken by microscopy, where it can be seen how the cells after the wound caused begin to migrate and this migration is increased in the presence of PDA: however, BV was harmful to cells, generating cell death, as shown in Table 4, where there was practically no cell migration. It has been reported that cell migration can be reduced during infection or cell aging (Kondo & Yonezawa, (1992). Changes in the migratory ability of human lung and skin fibroblasts during in vitro aging and in vivo cellular senescence. Mechanisms of ageing and development, 63: 223-233.). This process is increased when there is production of collagen and other extracellular matrix proteins.

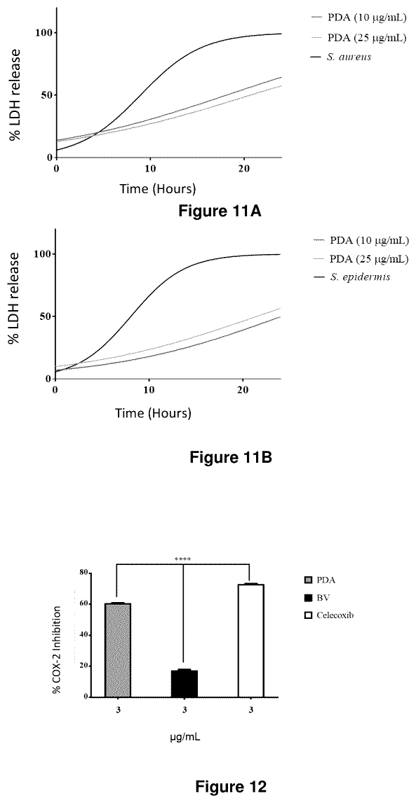

Example 5C: Reduction of bacterial Infection by measuring the release of the enzyme Lactate Dehydrogenase (LDH): The HDFa line was infected with S. aureus and S. epidermis using an MOI of 5, adding PDA at concentrations of 10 and 25 pg/mL, the release of the enzyme LDH was evaluated. LDH is a damage marker which is released when the cell membrane is damaged, using medium as negative control and positive control of LDH release using Triton X-100 detergent. It is observed that a lower release of LDH is obtained over time when the infection is carried out with S. epidermis and PDA FIG. 11B), which is related to the greater susceptibility of this bacterium to PDA; also, a decrease in LDH is observed when infected with S. aureus (FIG. 11A). Therefore, PDA provides a protective effect at the cellular level.

Example 5D: Anti-inflammatory activity. Tests were carried out to evaluate its anti-inflammatory potential, by inhibition of the enzyme COX-2. The results are shown in FIG. 12. A 60% inhibition of the activity of the COX-2 enzyme is obtained when using PDA and 20% inhibition when using 3 pg/mL of BV. Celecoxib (a drug that inhibits the pro-inflammatory action of COX-2) was used as a control. Consequently, PDA has very positive pro-inflammatory effects, which is important against epithelial damage, and BV has no effect.

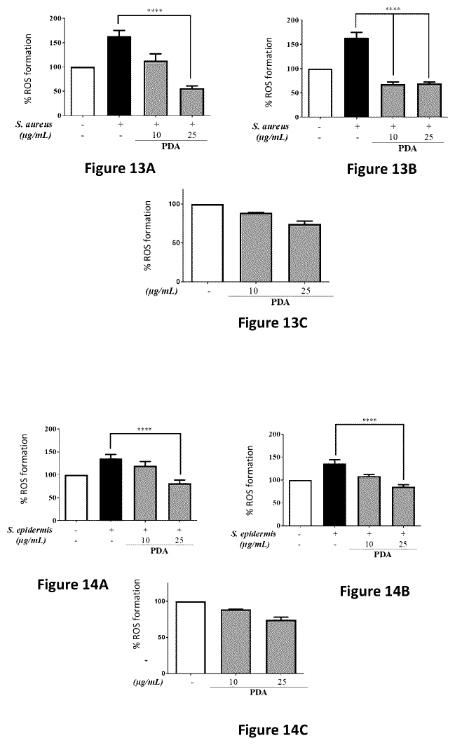

Example 5E: The effect of PDA on HFDa cells exposed to Infection induced by epithelial pathogens—S. epidermis and S. aureus—(FIGS. 13 and 14). These pathogens when they attack cells, generating an increase in free radicals at the intracellular level, triggering damage to macromolecules such as DNA, proteins and lipids and generating collagen destruction. PDA is capable of protecting fibroblasts from the harmful effect generated by pathogens that generate an increase in ROS and increase inflammation. This test could be extrapolated to other noxa such as UV rays. With which it shows that PDA has a powerful protective effect at the level of: stimulating migration, anti-inflammatory and reducing ROS (against bacterial noxa).

Example 6: Formulation of an anti-aging serum containing PDA and Apitop®. The present invention provides a purified bee venom (PDA) and its use as a key ingredient to delay antiaging, it also comprises a suitable cosmetic vehicle composition, without perfumes, and its color is given only by natural components. The PDA provides Mn, Ap, MCD and other molecules present in smaller quantities in the BV, such as sugars, amino acids, minerals. The PDA is presented in a concentration of 0.006-0.012% p/p, which is mixed with Apitop® at 10% p/p (propolis, royal jelly, pollen, honey and vitamins C and D), in addition, it contains hyaluronic acid, at 2% w/v and vitamin E.

The Serum contains a suitable concentration of PDA (0.006 to 0.012% w/w) for topical application on the skin; In addition, it contains Mn, a penetrating peptide that can help all the other components of the Serum to pass through the dermis and the bio-active components achieve their regenerative action, protective against ROS. The formulation of the Serum is soft, without perfumes and has a unique smell and color, given by its natural components. The advantages of this Serum is that it has components such as PDA plus Royal Jelly and propolis, which have been tested in vitro, showing that it has anti-radical species activity (ANTIROS), regenerative properties for the skin (anti-aging) and anti-inflammatory. As indicated in the state of the art, previous patents have used the complete BV or solvent extractions, but without removing the allergenic agents (KR101394817B1). In this invention they refer to the viability of epithelial pathogens that cause acne and in our case both PDA and BV have an effect on bacteria. Nevertheless; BV has a strong cytotoxicity on human epithelial cells, generating their death at concentrations indicated in said invention. Patent EP 2460 526A formulates a cosmetic preparation focused on treating acne based only on BV and in this case they use test concentrations in HEK epithelial cells of 0.01-10 g/mL of BV. Finally, this formulation contains 0.001% BV, however, the possibility of allergic reactions that they may generate is not clear.

Example 7: Formulation of Serum: The raw materials 1, 2, 3, 4 (Table 5) are added in the order indicated in Table 5 at a temperature of 22° C. and homogenized until the formation of a gel which is leave for 24 hours for stabilization. Potassium sorbate (5, see Table 5) is then added, which is a natural preservative and surfactant that allows, at a certain temperature, to join different phases, such as vitamin E (fat-soluble) with the other components, lowering the surface tension of the emulsion, then components 7 and 8 are added (Table 5). After that, component 9 (Apitop®) is added and mixed with homogenization until the appropriate consistency is obtained, for which the pH is maintained at 6. Finally, the aqueous solution of active component 11 (PDA) prepared at 1% in water is added, and the mixture is carried out with a homogenizer at very low speed, in order to avoid denaturation of the protein components of the formulation. All the mixture is made in amber containers and protected from light.

TABLE 5
Formulation Serum 0.006-0.012 PDA

| | Raw Material | % weight |
|---|---|---|
| 1 | Carbopol 940 | 0.52 |
| 2 | Distilled water | 81.93 |
| 3 | Microcare | 1.26 |
| 4 | Triethanolamine | 0.26 |
| 5 | Potassium sorbate | 0.026 |
| 6 | Oily vitamin E | 1 |
| 7 | Liponic EG-1 | 2 |
| 8 | Hyaluronic acid | 2 |
| 9 | Apitop ® | 10 |
| | Propolis extract | 3* |
| | Pollen | 2.5* |
| | Honey | 4.1* |
| 10 | Tween 80 | 1 |
| 11 | PDA (purified from bee venom) | 0.006-0.012 |
| | Apamine* | 0.0029442* (traces) |
| | Melitine* | 0.0079608* (traces) |
| | MCD* | 0.0015900* (traces) |
| | Minimine | Traces* |
| | Procamine A, B | Traces* |
| | Secarpine | Traces* |
| | Tertiapine | Traces* |
| | Melitine F | Traces* |
| | Cardiopep | Traces* |
| | Protease inhibitors | Traces* |
| | Noradrenaline | Traces* |
| | Dopamine | Traces* |
| | Histamine | Traces* |
| | Hydrocarbons (glucose, fructose) | Traces* |
| | Amino acids | Traces* |
| | Gamma Aminobutiric acid | Traces* |
| | Beta aminoisobutiric acid | Traces* |
| | Minerals (P, Ca, Mg) | Traces* |

Note:
The * indicates its composition of superior active ingredient (Propolis extract and purified apitoxin). Traces*: below the detection limit.

1. Method for preparing a purified bee venom comprising:
a) separating by molecular weight, the components present in bee venom (BV), solubilizing it in deionized water, and then filtering it through a 0.45 and 0.22 membrane pm to remove impurities, including particulate matter;
b) eliminate the phospholipase (PLA2) and hyaluronidase (HYA) components by ultrafiltration, and obtain a filtrate (FVB) containing MCD, Ap and Mn (<5% w/w), and free of PLA2 and HAY and a concentrate (CVB) comprising Mn (>90% of the total) and Ap (≥2% of the total) and traces or below the detection limit MCD and PLA2 and HYA;
c) recovering Mn, Ap and traces or below the MCD detection limit of said CVB obtained from step b), by chromatography on Silica gel (G60); and
d) resuspending said FVB obtained in step b) and said CVB resulting from the chromatography of step c), and storing at 4° C.

2. The method of claim 1 wherein said bee venom is selected from a bee venom powder preparation.

3. The method of claim 1 wherein said bee venom is obtained from a hive.

4. The method of claim 1 wherein in that step b) further comprising monitoring the elimination of PLA2 and HAY.

5. The method of claim 4 wherein in that said monitoring comprising performing enzymatic assays and reverse phase HPLC high performance chromatography.

6. The method of claim 1 wherein further comprising lyophilizing the purified bee venom obtained from step d).

7. The method of claim 1 in that step c) further comprising concentrating by rotary evaporation at 40° C.

8. Purified bee venom comprising apamin (Ap), melittin (Mn), MCD and trace elements including minimin, procamine A, B, secarpine, tertiapine, melittin F, cardiopep, protease inhibitors, norepinephrine, dopamine, histamine, carbohydrates including glucose and fructose, amino acids, gamma aminonobutyric acid, beta aminoisobutyric acid, minerals including phosphorus, calcium, magnesium.

9. Anti-aging serum formulation comprising a purified bee venom comprising Mn, Ap, MCD and selected trace elements of sugars, amino acids and minerals and a cosmetic vehicle composition comprising propolis and royal jelly, where the concentration of the purified bee venom is 0.006-0.012% w/w and where the concentration of the cosmetic vehicle composition is 10% w/w.

10. The formulation of claim 8 also comprising 2% w/v hyaluronic acid and vitamin E.
